(19) United States
(12) Patent Application Publication
(10) Pub. No.: US 2021/0375394 A1
(43) Pub. Date: Dec. 2, 2021

(54) GENE TO TRANSCRIPTOME ASSOCIATION PLATFORM FOR DRUG TARGET IDENTIFICATION

(71) Applicant: University of South Carolina, Columbia, SC (US)

(72) Inventors: Ioulia Chatzistamou, Irmo, SC (US); Hippokratis Kiaris, Irmo, SC (US); Youwen Zhang, Columbia, SC (US)

(73) Assignee: University of South Carolina, Columbia, SC (US)

(21) Appl. No.: 17/221,003

(22) Filed: Apr. 2, 2021

Related U.S. Application Data
(60) Provisional application No. 63/033,272, filed on Jun. 2, 2020.

(51) Int. Cl. G16B 25/10

(52) U.S. Cl. CPC G16B 25/10

(57) ABSTRACT

Systems, methods and diagnostic tools based a paradigm on how the diversity in expression profiles of primary specimens could be leveraged for target discovery via evaluating transcriptomes that lose coordination between the disease carrying and control groups and assessing the biological functions that are acquired in the former group.

### STATEMENT REGARDING FEDERALLY SPONSORED RESEARCH

This disclosure was made with the government support under OIA1736150 awarded by National Science Foundation. The government may have certain rights in the invention.

### TECHNICAL FIELD

The present disclosure relates to a paradigm on how the diversity in expression profiles of primary specimens could be leveraged for target discovery via evaluating transcriptomes that lose coordination between the disease carrying and control groups and assessing the biological functions that are acquired in the former group.

### BACKGROUND

Frailty is a clinical syndrome that is characterized by reduced responsiveness to stressors due to physiological decline in multiple organs and is associated with poor health outcomes including falls, incident disability, hospitalization, and mortality. Frailty is usually studied in the elderly, yet it affects younger individuals as well, 45-64 years old. With the number of Americans aged 65 and older projected to double by 2060, frailty is a condition with important implications in the quality of life of older individuals and overall healthcare management.

Despite that this condition is being recognized as a distinct clinical entity, our understanding of its pathogenetic mechanism remains limited. Differential expression analyses provide powerful tools for the identification of genes playing a role in disease pathogenesis. Yet, such approaches are usually restricted by the high variation in expression profiles when primary specimens are analyzed. Comprehensive molecular studies at the whole transcriptome level, were only recently initiated underscoring the role of a pro-inflammatory response in the development of this condition. Despite this progress, additional research is imperative, both at the level of generation of new primary experimental data and at the level of application of novel analytical approaches, facilitating extraction of biologically relevant and clinically meaningful information.

Conventionally, gene expression analyses aim to identify differentially expressed genes in predefined experimental groups. In such analyses, the magnitude of over- or under-expression is considered indicative for the impact of the corresponding genes in the pathology of interest. Such strategies are frequently limited by the variation in expression between specimens which is particularly relevant when genetically diverse specimens are analyzed.

Accordingly, it is an object of the present disclosure to overcome these limitations. The current disclosure has applied an alternative strategy in which samples were evaluated by comparing the correlation of expression of specific genes with the whole transcriptome, in different experimental groups. Coupling such analysis with publicly available gene ontology platforms could identify changes in the transcriptome that would not be appreciated by conventional differential expression analysis. Furthermore, it could provide hints regarding the biological implications of such changes. For example, by focusing on the unfolded protein response (UPR) we were able to unveil specific functions of UPR branches and how they change during pathology. It is conceivable that with the assessment of the degree of coordination in gene expression as opposed to the magnitude of differential expression, we may obtain hints underscoring different biological and pathological states.

Citation or identification of any document in this application is not an admission that such a document is available as prior art to the present disclosure.

### SUMMARY

The above objectives are accomplished according to the present disclosure by providing in a first embodiment, a transcriptome correlation method. The method may include calculating a composite correlation index that may include calculating a correlation coefficient value for each transcript with respect to every other transcript in a transcriptome via at least one pairwise comparison, the composite correlation index may indicate either coordination or abolishment of coordination for the at least one pairwise comparison. Further, a negative composite correlation index may show an inversed profile of gene coordination. Still, a positive composite correlation index may show gene coordination was maintained. Yet again, the composite correlation index may show an extent of transcriptional reprogramming. Moreover, the extent of transcriptional reprogramming may indicate a presence of a disease state. Further yet, the disease state may be steatosis. Still, a composite correlation index indicative value may indicate a pro-inflammatory response and transcriptional reprogramming even though no histopathological evidence of inflammation is present. Still yet, the method may identify genes exhibiting changes in their transcriptomic profile via calculation of a cumulative composite correlation index. Further yet, the cumulative composite correlation index may be calculated via adding independent composite correlation indexes of at least two pairwise comparisons. Still further, the method may include calculating a correlation coefficient value for each transcript with respect to every other transcript in a transcriptome via at least three pairwise comparisons that may include control versus steatosis, control versus non-steatosis, and steatosis versus non-steatosis.

In a further embodiment, an unbiased whole transcriptome analysis is provided. The analysis may include determining an extent of expression of all transcripts in an organ via coordination analysis, determining via at least one pairwise comparison an extent of transcriptome reorganization, and showing engagement of T cell activation to indicate a presence of a disease state. Further, the organ may be a liver. Still, the disease state may be steatosis. Moreover, a negative composite correlation index may show an inversed profile of gene coordination. Still yet, a positive composite correlation index may show gene coordination was maintained. Still, the composite correlation index may show an extent of transcriptional reprogramming. Furthermore, the extent of transcriptional reprogramming may indicate a presence of a disease state. Still further, a composite correlation index indicative value can indicate a pro-inflammatory response and transcriptional reprogramming even though no histopathological evidence of inflammation is present. Still further, the analysis may include identifying genes exhibiting changes in their transcriptomic profile via calculation of a cumulative composite correlation index. Moreover, the cumulative composite correlation index may be calculated via adding independent composite correlation indexes of at least two pairwise comparisons. Further, the analysis may include calculating a correlation coefficient value for each transcript with respect to every other transcript in a transcriptome via at least three pairwise comparisons: control versus steatosis, control versus non-steatosis, and steatosis versus non-steatosis.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows heatmaps of the correlation coefficients (R) among all pairwise comparisons between the most highly expressed genes in the NOR group.

FIG. 2 shows violin plots showing the R values between each of TSIX, BEST1, ADAMTSL4 and MAP3K13 in the NOR and the FRA groups.

FIG. 3 shows Function Retention Index (FRI) and Function Acquisition Index (FM) for each of TSIX, BEST1, ADAMTSL4 and MAP3K13.

FIG. 4 shows an outline of the coordination analysis applied in the present study.

FIG. 5 shows Table 1, Biological processes according to GO that were common for TSIX, BEST1 and ADAMTSL4 in the FRA group.

FIG. 6 shows response of deer mice (P. maniculatus) to HFD: body weight in genetically diverse P. maniculatus after administration of regular diet or HFD.

FIG. 7 shows histopathological appearance of liver sections (H&E) from animals that received regular diet (i) or HFD (ii) but did not develop steatosis or received HFD and developed moderate (iii) or severe (iv) steatosis.

FIG. 8 shows Pc calculation for the liver transcriptome of P. maniculatus fed with regular diet (C) or HFD and developed (S) or did not develop (NS) steatosis.

FIG. 9 shows the number of differentially expressed genes, the volcano plots and the top three upregulated and down regulated genes in all three pairwise comparisons. FDR cutoff is 0.1 and minimum fold change is 2.

FIG. 10 shows Table X—Table 1—Gene Ontology analysis based on Pc data.

FIG. 11 shows Table Y—Gene Ontology analysis based on differential expression.

The figures herein are for illustrative purposes only and are not necessarily drawn to scale.

### DETAILED DESCRIPTION OF A PREFERRED EMBODIMENT

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

The current disclosure analyzed a publicly available dataset related to frailty, a syndrome characterized by reduced responsiveness to stressors and exhibiting increased prevalence in the elderly. We evaluated the transcriptome that loses its coordination between the frailty and control groups and assessed the biological functions that are acquired in the former group. Among the top genes exhibiting the lowest correlation, at the whole transcriptome level, between the control and frailty groups were TSIX, BEST1 and ADAMTSL4. Processes related to immune response and regulation of cellular metabolism and the metabolism of macromolecules emerged in the frailty group. The proposed strategy confirms and extends earlier findings regarding the pathogenesis of frailty and provides a paradigm on how the diversity in expression profiles of primary specimens could be leveraged for target discovery.

Correlation analysis is used widely to identify genes exhibiting correlated expression and to infer regulatory associations between and among gene clusters. Coupling co-regulation analysis with function prediction tools possesses unique power in predicting gene function in a manner that is especially applicable to genetically diverse specimens. By using the unfolded protein response as a paradigm, we applied this strategy to evaluate biological activities for known genes, to assign them to particular UPR branches and ultimately to explore how such associations are altered in pathology providing targets for drug development.

Differential expression analyses provides a powerful strategy to identify disease-related genes and unveil molecular targets for drug development. Such approach though suffers when specimens from genetically diverse individuals are analyzed. In order to address this limitation we developed a strategy that instead of differential expression it focuses on the coordination of gene transcripts at the whole transcriptome level, and of its loss when disease emerges. With this strategy genes that abolish or gain coordination with a transcriptome, specifically in disease, emerge as targets for drug development.

Data accumulation through genomic and transcriptomic analyses require novel tools/strategies for the extraction of biologically relevant and clinically useful information. A major limitation of existing approaches is that they focus on the magnitude of differential expression between groups, a strategy that is restricted when specimens from human, genetically diverse populations are studied. We developed a strategy that extracts meaningful information relying on the evaluation of coordination in gene expression and of its loss (or gain) in disease. This strategy is capable of identifying disease relevant targets even when their expression is minimally induced or suppressed in disease. We have applied this approach in publicly available data and data and generated by us on liver disease, and on publicly available data on the frailty syndrome in people. The validity of our strategy has been confirmed by the identification, in all cases of gene targets that are known to operate as such. In addition, a list of novel targets for hepatic steatosis has been discovered.

Among the gene targets discovered by our strategy for the treatment of hepatic steatosis and other related conditions are: (1) Sema4d—For this gene blocking antibodies developed for cancer management (by Vaccinex); (2) Vav1—An inhibitor is already available named Azathioprine (Imuran) for rheumatoid arthritis, granulomatosis with polyangiitis, Crohn's disease, ulcerative colitis, systemic lupus erythematosus, and in kidney transplants; and (3) Sox18—An inhibitor is already available named R(+)-propranolol [Inderal (Beta blocker)-racemic mixture or R(+) and S(−) enantiomers/reportedly the S(−) is more potent than the R(+) for the indicated conditions, high blood pressure, irregular heart rate, thyrotoxicosis, capillary hem angiomas, performance anxiety, and essential tremors].

By arbitrarily selecting at least 70 reads as the cut-off in the NOR group we identified 178 highly expressed transcripts. This limit was set for the convenience of the calculations and in theory could be increased indefinitely, provided that appropriate tools for computational analysis are developed. For the same reason specimens were assigned to only 2 groups, the NOR and the FRA groups. However additional sub-groups could be utilized, if a higher number of samples were available.

Initially, we asked how the expression among these 178 highly expressed genes is correlated between the NOR and FRA groups. To that end, we calculated the correlation coefficient R (Pearson's) for all pairwise comparisons between these 178 highly expressed genes, generating a heatmap illustrating the correlation in their expression.

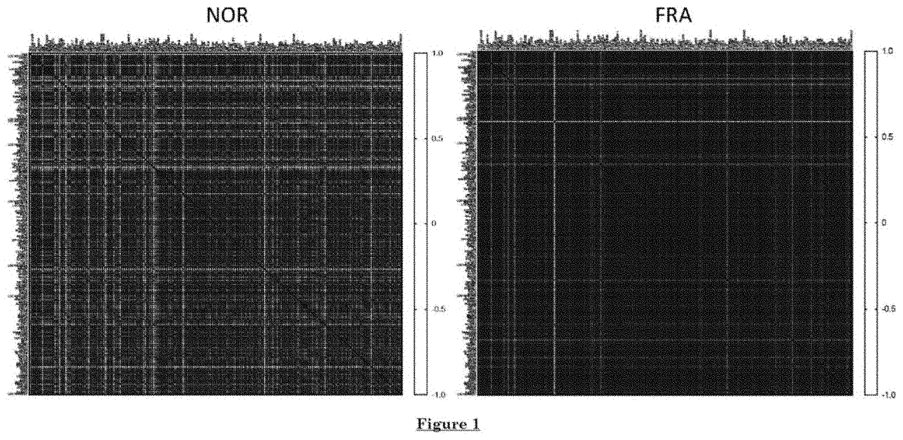

As shown in FIG. 1, the vast majority of the genes subjected to this type of analysis was highly correlated with each other and the correlation increased in the FRA group. FIG. 1 shows Heatmaps of the correlation coefficients (R) among all pairwise comparisons between the most highly expressed genes in the NOR group. It is generally accepted that correlated expression or co-expression implies co-regulation, by the same or similar transcription factors that define transcriptional networks. According to the results of FIG. 1, this co-regulation becomes more intense during frailty. It is plausible that the lower degree of correlation in the control group (NOR) is indicative of the margins of expression at which physiological function for these genes can be attained. This flexibility is abolished in frailty because activation of signaling pathways under these conditions dictates more robust expression profiles. Correlation was more intense in primary fibroblasts of outbred rodents, under endoplasmic reticulum stress as compared to unstressed cells in culture.

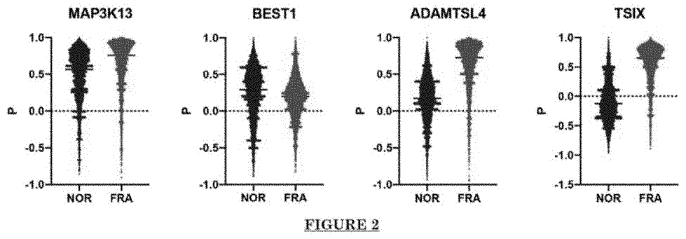

Subsequently, we estimated how the whole transcriptome is correlated with these 178 genes and compared how this correlation changes during frailty. To that end, a composite correlation (Pc) was calculated for each gene which corresponds to the correlation of the R values this gene has, with the whole transcriptome between the NOR and FRA groups. Then, we ranked these genes according to Pc. Therefore, high Pc indicates retention of coordination between the NOR and FRA groups while low Pc is suggestive for the loss of coordination, when the pathology emerges. The top 3 genes with lowest Pc were TSIX, BEST1 and ADAMTSL4 (−0.069, 0.074 and 0.135 respectively) while the top 3 with highest Pc were PNPT1, ORAI2 and MAP3K13 (0.462, 0.462 and 0.466 respectively), see FIG. 2. FIG. 2 shows violin plots showing the R values between each of TSIX, BEST1, ADAMTSL4 and MAP3K13 in the NOR and the FRA groups. These genes, such as TSIX, BEST1 and ADAMTSL4, are the ones that according to our hypotheses are being affected by (or affecting) frailty, or being affected minimally by this syndrome, such as PNPT1, ORAI2 and MAP3K13. TSIX encodes for an antisense RNA that is involved in the regulation of XIST and therefore in X chromosome inactivation. BEST1 encodes for a member of the bestrophin family of proteins that are calcium-activated chloride channels and have been associated with retinal disease. ADAMTSL4 participates in the formation of microfibrils and is associated with the development of ectopia lentis, an eye disorder.

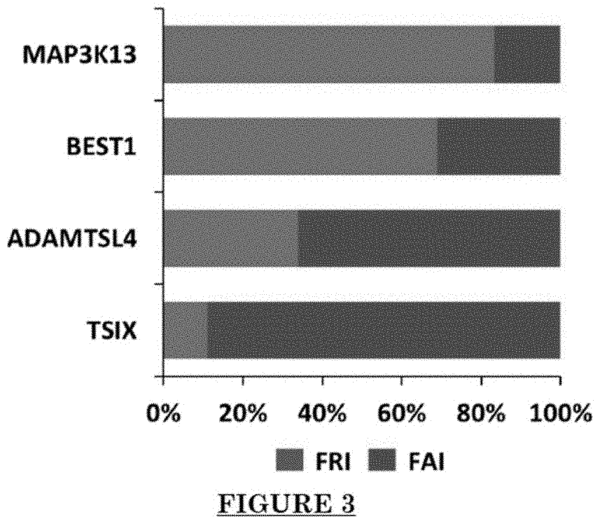

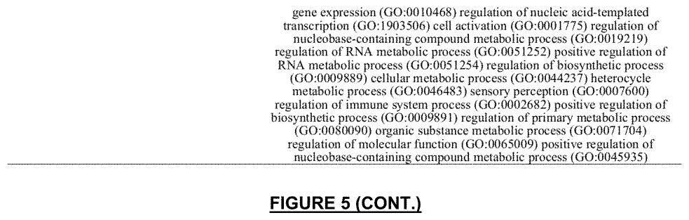

In order to better understand the relevance of loss of coordination in TSIX, BEST1 and ADAMTSL4 we ranked the transcriptome according to its coordination with these 3 genes, Then, by using R=0.5 as a cut-off, we subjected the corresponding transcriptome to GO analysis. This analysis indicated that for the same gene, several functions were retained between the NOR and FRA groups, but several novel functions were also acquired, see FIG. 3. FIG. 3 shows Function Retention Index (FRI) and Function Acquisition Index (FAI) for each of TSIX, BEST1, ADAMTSL4 and MAP3K13. FRI reflects the ratio of the functions in the NOR group that were retained in the FRA group (FRI=common functions in both groups/all functions in FRA group). FAI reflects the ratio of the novel functions in the FRA group that were absent from the NOR group (FAI=new functions in FRA group/all functions in FRA group). Among the latter, the most prominent ones included functions related to immune system processes and metabolic processes, see FIG. 5, Table 1, Biological processes according to GO that were common for TSIX, BEST1 and ADAMTSL4 in the FRA group.

These findings confirm and extend previous findings on the role of immune system in the pathogenesis of frailty. They also identify the significance of metabolic deregulation or reprogramming in the development of this syndrome. In addition, they provide novel gene targets that may play a role in the development of this condition. It is conceivable that refinement of the proposed strategy, by including larger datasets and deeper and more expanded roster of genes to initiate the co-regulation assessment, will be applicable to various conditions and be leveraged—as opposed to be restricted—by the high variation, when genetically diverse specimens are analyzed.

Materials and Methods

Data used were retrieved from GEO (Accession number: GSE129534). Specimens' characteristics are described in detail in the original study [9]. Participants of the study were from the Healthy Aging in Neighborhoods of Diversity across the Life Span (HANDLS) study of the National Institute on Aging Intramural Research Program (NIA IRP), National Institutes of Health. In our analysis we assigned the specimens in 2 groups, with (FRA) or without (NOR) frailty, consistently with the classification of the original study Prince C S, Noren Hooten N, Mode N A, Zhang Y, Ejiogu N, Becker K G, Zonderman A B, Evans M K. Frailty in middle age is associated with frailty status and. race-specific changes to the transcriptome. Aging (Albany N.Y.). 2019; 11:5518-34. https://doi.org/10.18632/aging.102135 PMID:31395793. Each group consisted of 8 samples, each of which included 4 whites and 4 African Americans, both males (50%) and females (50%). All individuals were 45-49 years old (Mean±sd=48.09±1.21 and 47.85±1.84 for the NOR and FRA groups respectively). RNA-seq was performed in peripheral blood mononuclear cells, Id.

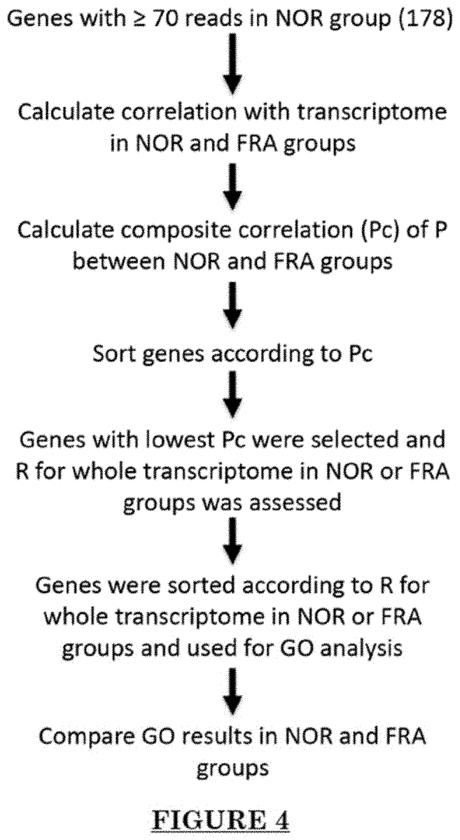

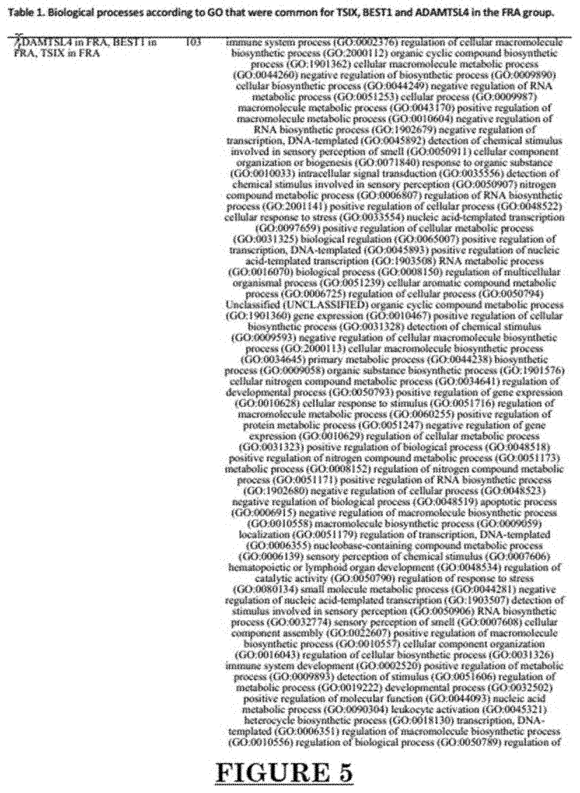

The experimental outline we applied is shown in FIG. 4. FIG. 4 shows an outline of the coordination analysis applied in the present study. Initially we identified the transcripts exhibiting relatively high abundance. Arbitrarily we selected genes that displayed at least 70 reads in the NOR group (resulting in n=178 highly expressed genes). Subsequently we calculated the correlation (R, Pearson's) for these 178 genes with the whole transcriptome, independently in the NOR and the FRA groups. In order to test for which of these genes correlation with the transcriptome changes in the different groups, we calculated the Pc from the R values calculated above. This transformation assigned a unique Pc value to each of these genes which reflects the degree by which coordination with the whole transcriptome changes in the 2 groups for the corresponding genes of interest. Then, the genes were sorted according to Pc, and for the ones that exhibited the lowest Pc (3 genes in this study) their correlation (R) with the whole transcriptome, in the NOR and the FRA groups was calculated. These R values were used to sort the transcriptome and supply it to a GO platform for further analysis. As a cut-off we arbitrarily chose genes with R>0.5. Finally, predicted functions were compared between the NOR and the FRA groups for the genes selected.

The induction of inflammation characterizes the transition of hepatic steatosis to non-alcoholic steatohepatitis. By applying a novel strategy, involving correlation of each transcript with every other in the transcriptome of outbred deer mice that received high fat diet, we show that transcriptional reprogramming directing immune cell engagement proceeds robustly even in the absence of steatosis. In the liver transcriptomes of animals with steatosis, a preference for the engagement of regulators of T cell activation was also recorded as opposed to the steatosis-free livers at which non-specific lymphocytic activation was seen. These analyses also revealed that as compared to controls, in the animals with steatosis, transcriptome coordination was subjected to more widespread reorganization while in the animals without steatosis reorganization was less extensive. None of these changes could be recorded by conventional differential expression analysis that only revealed enrichment of genes related to lipid metabolism. This highly versatile strategy suggests that the molecular changes inducing inflammation proceed robustly even before any evidence of steatohepatitis is recorded, either histologically or by differential expression analysis.

Non-alcoholic steatohepatitis (NASH) develops in livers that have accumulated histopathological changes associated with hepatic steatosis and are reflected to the differential expression of genes linked to the induction of inflammation. See, Schuster S, Cabrera D, Arrese M, Feldstein A E. Triggering and resolution of inflammation in NASH. Nat Rev Gastroenterol Hepatol. 2018:349-364, Parthasarathy G, Revelo X, Malhi H. Pathogenesis of Nonalcoholic Steatohepatitis: An Overview. Hepatol Commun. Jan. 14, 2020; and Cohen J. C., Horton J. D., Hobbs H. H. Human fatty liver disease: old questions and new insights. Science. 2011; 332:1519-1523. During disease progression extensive transcriptional reprogramming occurs that underscores its different stages. This multistage process that can be recapitulated with relatively high accuracy in animal models receiving special diets, alone or combined with other stimuli triggering liver injury (4). See, Farrell G, Schattenberg J M, Leclercq I, Yeh M M, Goldin R, Teoh N, Schuppan D. Mouse Models of Nonalcoholic Steatohepatitis: Toward Optimization of Their Relevance to Human Nonalcoholic Steatohepatitis. Hepatology. 2019 May; 69(5):2241-2257. Among them, outbred models may be of special value since they can mimic the different courses of disease progression in human patients at which steatosis develops stochastically. Havighorst A, Zhang Y, Farmaki E, Kaza V, Chatzistamou I, Kiaris H. Differential regulation of the unfolded protein response in outbred deer mice and susceptibility to metabolic disease. Dis Model Mech. Feb. 27, 2019; 12(2).

Essential for the molecular characterization of different subtypes of liver disease is differential expression which reveals specific transcripts that are enriched or depleted at different disease stages. See Hoang, S. A., Oseini, A., Feaver, R. E. et al. Gene Expression Predicts Histological Severity and Reveals Distinct Molecular Profiles of Nonalcoholic Fatty Liver Disease. Sci Rep 9, 12541 (2019), Bertola A, Bonnafous S, Anty R, et al. Hepatic expression patterns of inflammatory and immune response genes associated with obesity and NASH in morbidly obese patients. PLoS One. 2010; 5(10), and Morrison M C, Kleemann R, van Koppen A, Hanemaaijer R, Verschuren L. Key Inflammatory Processes in Human NASH Are Reflected in Ldlr−/−. Leiden Mice: A Translational Gene Profiling Study. Front Physiol. 2018; 9:132. Such quantitative changes in expression usually illuminate full-fledged pathology while subtle alterations, despite their potential significance may remain unnoticeable. Evaluation of the coordination profile of the whole liver transcriptome at different disease stages may provide hints regarding the underlying molecular changes that conventional, differential expression analysis cannot. Such changes in coordination profile appeared relevant in characterizing different liver pathology stages by focusing on the unfolded protein response. See Zhang Y, Chatzistamou I, Kiaris H. Coordination of the unfolded protein response during hepatic steatosis identifies CHOP as a specific regulator of hepatocyte ballooning. Cell Stress Chaperones. Jun. 23, 2020; Soltanmohammadi E, Farmaki E, Zhang Y, Naderi A, Kaza V, Chatzistamou I, Kiaris H. Coordination in the unfolded protein response during aging in outbred deer mice. Experimental Gerontology. Dec. 5, 2020; 144:111191; and Zhang Y, Lucius M D, Altomare D, Havighorst A, Farmaki E, Chatzistamou I, Shtutman M, Kiaris H. Coordination Analysis of Gene Expression Points to the Relative Impact of Different Regulators During Endoplasmic Reticulum Stress. DNA Cell Biol. 2019 September; 38(9):969-981. Furthermore, such analysis applied to the most highly expressed genes in the transcriptome was shown capable of illustrating changes in patients with frailty syndrome Zhang Y, Chatzistamou I, Kiaris H. Identification of frailty-associated genes by coordination analysis of gene expression. Aging (Albany N.Y.). 2020.

In the present study we evaluated how the liver transcriptome is collectively reorganized in specimens with or without steatosis. Our studies were based on the premise that genes belonging to the same transcriptional networks are co-expressed and when pathology emerges the profile of coexpression is collective changed. See, Stuart, Joshua M; Segal, Eran; Koller, Daphne; Kim, Stuart K (2003). A gene-coexpression network for global discovery of conserved genetic modules. Science. 302 (5643): 249-55; Roy S, Bhattacharyya D K, Kalita J K. Reconstruction of gene co-expression network from microarray data using local expression patterns. BMC Bioinformatics. 2014; 15 Suppl 7(Suppl 7): S10; Luo J, Xu P, Cao P, Wan H, Lv X, Xu S, Wang G, Cook M N, Jones B C, Lu L, Wang X. Integrating Genetic and Gene Co-expression Analysis Identifies Gene Networks Involved in Alcohol and Stress Responses. Front Mol Neurosci. Apr. 5, 2018; 11:102; van Dam S, Võsa U, van der Graaf A, Franke L, de Magalháes J P. Gene co-expression analysis for functional classification and gene-disease predictions. Brief Bioinform. Jul. 20, 2018; 19(4):575-592; Amar D, Safer H, Shamir R. Dissection of regulatory networks that are altered in disease via differential co-expression. PLoS Comput Biol. 2013; 9(3); Kostka D, Spang R. Finding disease specific alterations in the co-expression of genes. Bioinformatics. Aug. 4, 2004; 20 Suppl 1:i194-9; and Hu R, Qiu X, Glazko G, Klebanov L, Yakovlev A. Detecting intergene correlation changes in microarray analysis: a new approach to gene selection. BMC Bioinformatics. Jan. 15, 2009; 10:20. For our studies we used outbred deer mice (Peromyscus) that upon high fat diet (HFD) administration develop steatosis at an incidence of about 50%. See, Havighorst, A., Crossland, J., and Kiaris, H. (2017). Peromyscus as a model of human disease. Semin Cell Dev Biol 61, 150-155. To address the coordination profile, we calculated the composite correlation for each gene in the transcriptome with every other gene and compared it in the controls, the animals that received HFD but did not develop steatosis and the animals that received HFD but developed steatosis. The results were coupled to gene ontology (GO) analyses. See, Ashburner M, Ball C A, Blake J A, Botstein D, Butler H, Cherry J M, Davis A P, Dolinski K, Dwight S S, Eppig J T, Harris M A, Hill D P, Issel-Tarver L, et al, and The Gene Ontology Consortium. Gene ontology: tool for the unification of biology. Nat Genet. 2000; 25:25-29 to reveal transcripts that more prominently abolished their coordination with the whole transcriptome. Our results, besides describing the overall coordination profile of the transcriptome at different conditions, showed that HFD triggers a robust induction of an inflammatory response, irrespectively of the onset of steatosis. This change was not apparent by conventional analyses of the differentially expressed transcripts. Furthermore it showed that what differentiates the liver transcriptomes with and without steatosis is the preference of the former for T cell activation and engagement of genes involved in cell cycle regulation.

Results

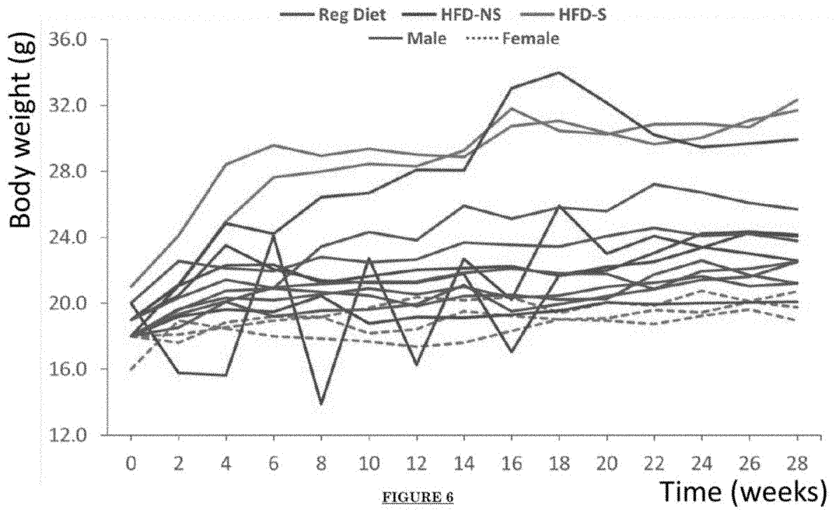

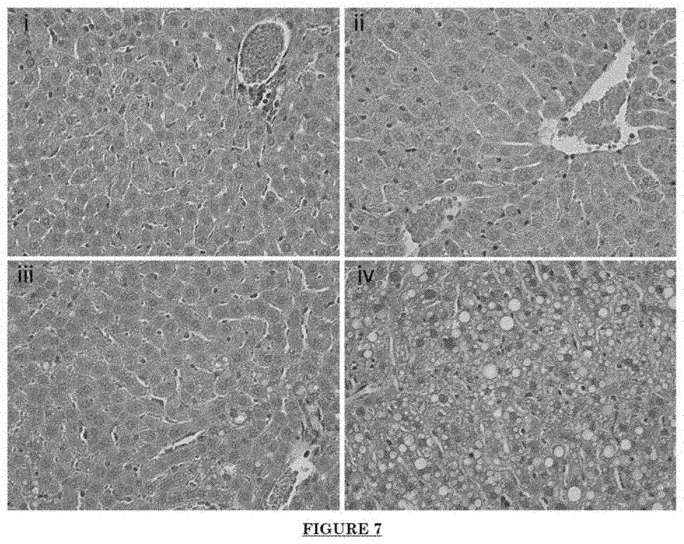

Variable response to HFD in outbred deer mice. A panel of 3-4 months old outbred deer mice (P. maniculatus) received HFD for about 6 months (n=10). Six animals received regular diet. Body weight was increased in the animals that received the HFD but remained highly variable, consistently with the genetically diverse nature of the experimental animals, see FIG. 6. Histology revealed the presence of steatosis in 5 out of 10 animals that received the HFD, see FIG. 7. No evidence of ballooning degeneration or lobular inflammation was recorded. See, Lackner C, Gogg-Kamerer M, Zatloukal K, Stumptner C, Brunt E M, Denk H. Ballooned hepatocytes in steatohepatitis: the value of keratin immunohistochemistry for diagnosis. J Hepatol. 2008 May; 48(5):821-8; Epub Feb. 22, 2008. PMID: 18329127; Brown G T, Kleiner D E. Histopathology of nonalcoholic fatty liver disease and nonalcoholic steatohepatitis. Metabolism. 2016; 65(8):1080-1086; and Matteoni C A, Younossi Z M, Gramlich T, Boparai N, Liu Y C, McCullough A J. Nonalcoholic fatty liver disease: a spectrum of clinical and pathological severity. Gastroenterology. 1999 June; 116(6):1413-9, suggesting that under these conditions the disease has not progressed to more advanced stages of non-alcoholic liver steatohepatitis (NASH), see FIG. 7.

Distinct profile of expression coordination in livers with or without steatosis. RNAseq was performed in the liver of P. maniculatus that received regular diet or HFD and results have been deposited to NCBI (GSE146846). To test how the transcriptome in each group is coordinated at these conditions we calculated the composite correlation (Pc) index as follows: Initially we calculated the correlation coefficient (R, Pearson's) for each transcript with every other transcript in the transcriptome, in all 3 pairwise comparisons being control vs steatosis (C vs S), control vs non-steatosis (C vs NS) and steatosis vs non steatosis (S vs NS) (See, Supplementary tables 1-3 Zhang, Chatzistamou, and Kiaris, Transcriptomic coordination at hepatic steatosis indicates robust immune cell engagement prior to inflammation. University of South Carolina. www.kiarislab.com). Pc of each transcript reflected the composite correlation coefficient of all correlation coefficients calculated above, for each given transcript, in the 3 pairwise comparisons (C vs S, C vs NS, and S vs NS). Therefore, high Pc values indicate that coordination is retained for the given comparison for the corresponding gene, while lower Pc values indicate abolishment of coordination. Conversely, negative Pc values suggest that the profile of coordination is inversed.

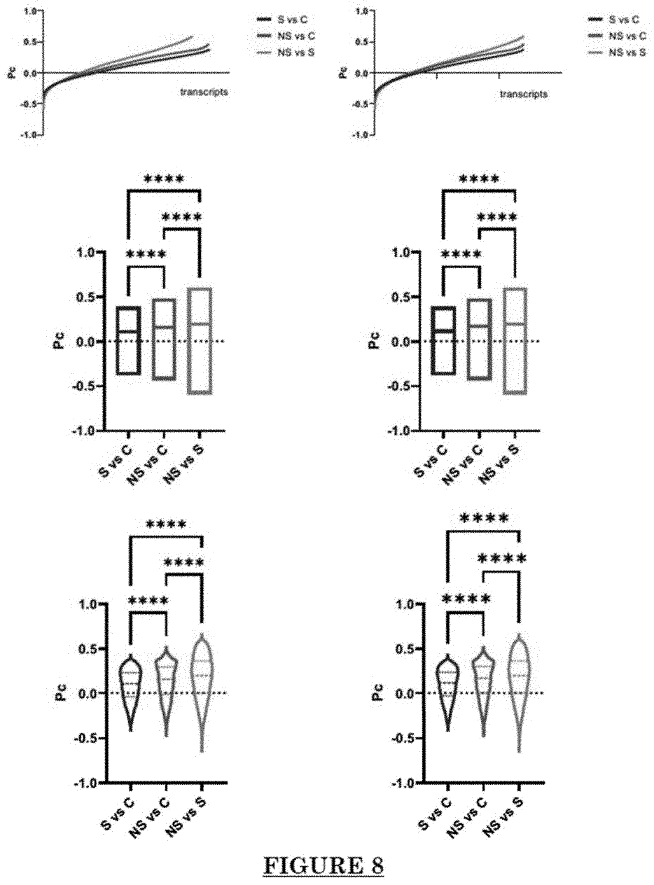

As shown in FIG. 8, in all three groups, the majority of the transcripts exhibited positive Pc values, suggesting that the mode of coordination was retained between the animals with or without steatosis for most of the genes. Higher Pc values (average Pc=0.17) were seen in the S vs NS groups suggesting that most genes retained their coordination upon HFD administration and irrespectively of the development of steatosis (see FIGS. 6 and 7). Conversely, lowest Pc (=0.086) was seen in the comparison between S and C suggesting extensive transcriptional reprogramming. In the comparison between NS and C average Pc had intermediate magnitude (Pc=0.13). All differences were statistically significant (P<0.0001). Similar were the findings when instead of the whole transcriptome only genes common in the 3 groups were evaluated suggesting that the findings do not reflect a bias towards transcripts that are present only in some experimental groups.

These results suggest that during HFD administration, extensive reprogramming of the transcriptome occurs, which is more pronounced in the livers that develop steatosis as compared to those that did not. The differences in the transcriptomic profile between the livers that did and those that did not develop steatosis at HFD, were more modest. Thus, special diet such as HFD induces more changes in the transcriptome than the pathology (steatosis) per se.

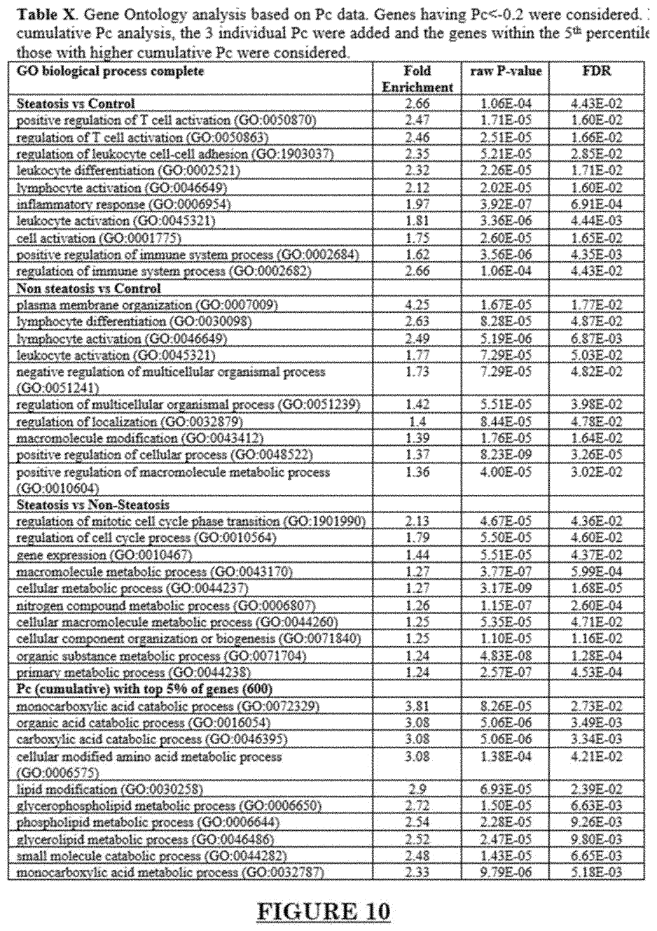

Gene Ontology analyses reveal engagement of inflammation by HFD. To obtain insights regarding the biological processes that are enriched for the transcripts exhibiting the most pronounced changes in Pc values in the 3 comparison groups we utilized the publicly available Gene Ontology Platform (Gene Ontology http://geneontology.org/), which is hereby incorporated by reference. For this analysis, the Pc values were sorted for each group in descending order and the genes exhibiting Pc<−0.2 were analyzed (Suppl. Table 4, Zhang, Chatzistamou, and Kiaris, Transcriptomic coordination at hepatic steatosis indicates robust immune cell engagement prior to inflammation. University of South Carolina. www.kiarislab.com). The results for the top 10 processes are shown in Table X, see FIG. 10 and were derived by using 752 genes, 700 genes and 854 genes for the S vs C, the NS vs C and the S vs NS genes respectively. Both comparisons involving administration of HFD (S and NS) vs C exhibited an enrichment for processes associated with a proinflammatory response. Thus, robust transcriptional reprogramming, consistent with the induction of inflammation, occurs irrespectively of steatosis and despite that no histopathological evidence of inflammation was seen. Comparison between the S vs NS specimens revealed that the most prominent processes were associated with regulation of cell cycle.

In order to identify genes that collectively exhibit changes in their transcriptomic profile across the different groups we calculated a cumulative Pc index by adding the 3 independent Pc indices for the genes that were common between the 3 individual pairwise comparisons. Then we sorted the genes in descending order according to their cumulative Pc and selected the top 5% which corresponded to about 600 genes for GO analysis (Table 1 and Suppl Table 5, Zhang, Chatzistamou, and Kiaris, Transcriptomic coordination at hepatic steatosis indicates robust immune cell engagement prior to inflammation. University of South Carolina. www.kiarislab.com). Not surprisingly, GO analysis indicated that processes associated with lipid metabolism were more prominently enriched.

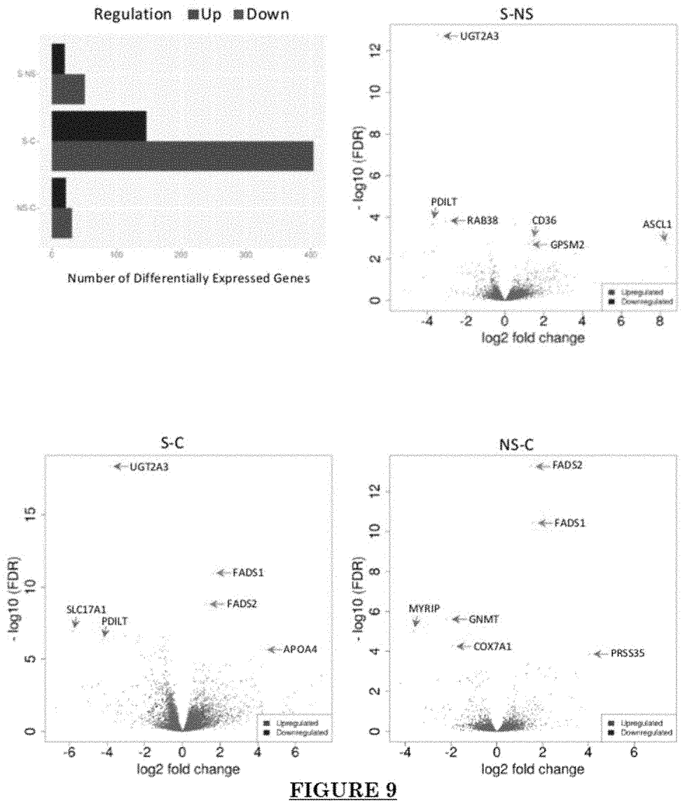

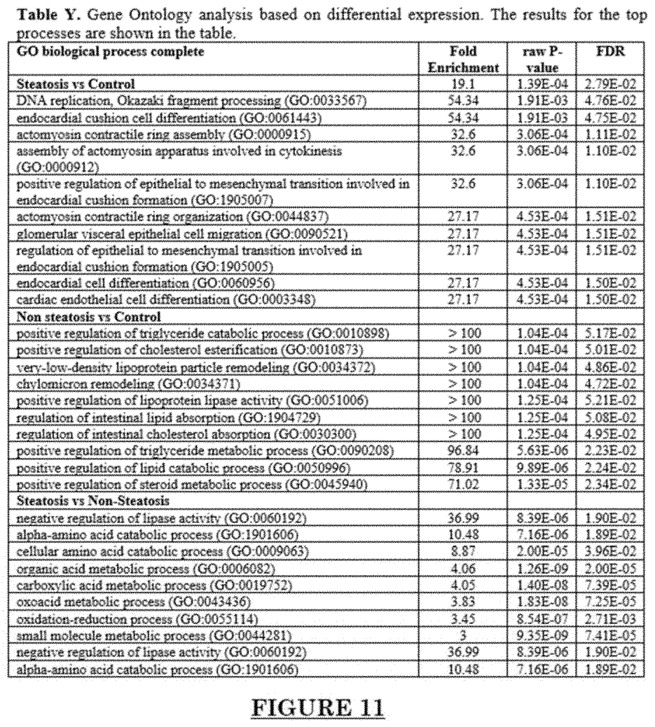

Differential expression only reveals a fraction of processes linked to steatosis. To appreciate the discovery power of the proposed coordination approach we also performed conventional differential expression and GO analysis (Suppl Table 6, Zhang, Chatzistamou, and Kiaris, Transcriptomic coordination at hepatic steatosis indicates robust immune cell engagement prior to inflammation. University of South Carolina. www.kiarislab.com). In both NS vs C and S vs NS, the processes that were found to be enriched were associated with lipid metabolism while in the S vs C comparison, processes associated with DNA replication were revealed (See FIG. 11, Table Y). The number of differentially expressed genes and the top 3 upregulated and downregulated transcripts in each comparison are shown in FIG. 9.

Discussion

The assessment of differential expression is an important indicator of genes associated with pathology however its value can be limited when genetically diverse specimens are analyzed. Genes highly relevant to disease may remain masked if the variation in gene expression between individuals reduce the statistical power of differential expression studies. For example, in hepatic steatosis, despite the established role of endoplasmic reticulum stress in disease development, genes associated with the unfolded protein response are not usually detected by differential expression analysis, see Hoang, S. A., Oseini, A., Feaver, R. E. et al. Gene Expression Predicts Histological Severity and Reveals Distinct Molecular Profiles of Nonalcoholic Fatty Liver Disease. Sci Rep 9, 12541 (2019). Such role though can be revealed when their coordination with the whole transcriptome is examined, see Zhang Y, Chatzistamou I, Kiaris H. Coordination of the unfolded protein response during hepatic steatosis identifies CHOP as a specific regulator of hepatocyte ballooning. Cell Stress Chaperones. Jun. 23, 2020. When the role of inflammation is studied in liver disease it marks only its more advanced stages and is frequently dissociated from steatosis, especially in some animal models. See, Rodriguez-Suarez E., Mato J. M., Elortza F. (2012) Proteomics Analysis of Human Nonalcoholic Fatty Liver, and In: Josic D., Hixson D. (eds) Liver Proteomics. Methods in Molecular Biology (Methods and Protocols), vol 909. Humana Press, Totowa, N J, Nassir F, Rector R S, Hammoud G M, Ibdah J A. Pathogenesis and Prevention of Hepatic Steatosis. Gastroenterol Hepatol (N Y). 2015; 11(3):167-175, Zijona E, Hijona L, Arenas J I, Bujanda L. Inflammatory mediators of hep atic steatosis. Mediators Inflamm. 2010; 2010:837419, and Wang, W., Xu, M.-J., Cai, Y., Zhou, Z., Cao, H., Mukhopadhyay, P., Pacher, P., Zheng, S., Gonzalez, F. J. and Gao, B. (2017), Inflammation is independent of steatosis in a murine model of steatohepatitis. Hepatology, 66: 108-123. While the deregulation of pro-inflammatory cytokines is detected in benign steatosis in the absence of typical liver inflammation they are generally considered as the direct outcome of aberrant lipid metabolism, occasionally originating from visceral fat, are linked to insulin resistance and are not representative of an orchestrated inflammatory response occurring in the liver. See, Bradbury M W. Lipid Metabolism and Liver Inflammation. I. Hepatic fatty acid uptake: possible role in steatosis. Am J Physiol Gastrointest Liver Physiol 290: G194-G198, 2006; Browning J D, Horton J D. Molecular mediators of hepatic steatosis and liver injury. J Clin Invest. 2004 July; 114(2):147-52; Targher, G., Bertolini, L., Scala, L., Zoppini, G., Zenari, L. and Falezza, G. (2005), Non-alcoholic hepatic steatosis and its relation to increased plasma biomarkers of inflammation and endothelial dysfunction in non-diabetic men. Role of visceral adipose tissue. Diabetic Medicine, 22: 1354-1358.

By using a novel unbiased whole transcriptome analysis, that relies on the extent of expression of all transcripts in livers from outbred rodents that developed or not steatosis after HFD administration, we were able to show that both in the specimens that did not show pathology and those that exhibited steatosis, a robust engagement of proinflammatory processes occurred. What however differentiated the two entities was the engagement of T cell activation processes that was detected only in the fatty livers. Conventional differential expression analysis that focuses on transcripts exhibiting quantitative differences in the experimental groups, failed to reveal any evidence of immune cell activation. Probably this limitation is related to the genetically diverse nature of the specimens in combination with the fact that such changes may be below the thresholds of significance of such analysis. Yet, coordination analysis, especially at the whole transcriptome level, leverages such diversity in gene expression among individual specimens and is capable of extracting meaningful information even when subtle changes occurred.

This coordination analysis also indicated in pairwise comparisons that a major difference of the livers with and without steatosis, as compared to the controls is that in those with steatosis, the transcriptome underwent more extensive reorganization compared to those without. Comparison though of the two, exhibited the higher retention in the profile of coordination. This suggest that diet supersedes pathology in shaping the profile of the transcriptome.

Differential analysis of gene expression was only able to reveal the enrichment of processes related to lipid metabolism in NS vs C and S vs NS comparison while comparison of S vs C was able to demonstrate engagement of pathways guiding DNA replication and Okazaki fragment processing.

Collectively, these results suggest that inflammatory engagement is robustly triggered by HFD even before inflammation is detectable in the histopathological analysis or by the differential expression studies, and illustrate the power of the proposed gene coordination approach to reveal changes that conventional strategies cannot.

Animals. Deer mice, P. maniculatus were obtained from the Peromyscus Genetic Stock Center (PGSC), University of South Carolina (USC), Columbia, S.C. (RRID:SCR_002769). Deer mouse, P. maniculatus bairdii (BW Stock), was closed colony bred in captivity since 1948 and descended from 40 ancestors wild-caught near Ann Arbor, Mich. Deer mice were fed either a regular chow diet or a high fat diet (HFD, 58 kcal % fat and sucrose, Research Diets D12331) for 28 weeks, starting at 3-4 months of age. Body weight was measured every two weeks. Animals were then sacrificed using isoflurane as an anesthetic followed by cervical dislocation, and the livers were collected. All animal procedures were approved by the Institutional Animal Care and Use Committee (IACUC) and the Department of Health and Human Services, Office of Laboratory Animal Welfare, University of South Carolina (Approval No. 2349-101211-041917).

RNA sequencing. RNA and library preparation, sequencing, and postprocessing of the raw data and data analysis were performed by the USC CTT COBRE Functional Genomics Core. RNAs were extracted with a Qiagen RNeasy Plus Mini kit as per manufacturer's recommendations (Qiagen, Valencia, Calif.). RNA integrity was assessed using the Agilent Bioanalyzer and samples had a quality score≥8.6. RNA libraries were prepared using established protocol with NEBNext Ultra II Directional RNA Library Prep Kit for Illumina (NEB, Lynn, Mass.). Each library was made with one of the TruSeq barcode index sequences and pooled together into one sample to be sequenced on the HiSeq 2×150 bp pair-ended sequencing platform (Genewiz). Sequences were aligned to the P. maniculatus genome (HU_Pman_2.1 (GCA_003704035.1)) in ensembl.org using STAR v2.7.2 (see Dobin, A., Davis, C. A., Schlesinger, F., Drenkow, J., Zaleski, C., Jha, S., et al. (2013). STAR: ultrafast universal RNA-seq aligner. Bioinformatics 29, 15-21). Reads were counted using the featureCounts function of the Subreads package (see Liao, Y., Smyth, G. K., Shi, W. (2013). The Subread aligner: fast, accurate and scalable read mapping by seed-and-vote. Nucleic Acids Res 41, e108.) using Ensembl GTF and summarized at exon, transcript, or gene level. Only reads that were mapped uniquely to the genome were used. The differentially expressed gene analysis was conducted with iDEP.91 (iDEP Platform http://bioinformatics.sdstate.edu/idep/) (see Ge, S. X., Son, E. W. & Yao, R. iDEP: an integrated web application for differential expression and pathway analysis of RNA-Seq data. BMC Bioinformatics 19, 534 (2018).)

Histology. Upon termination of the study the liver of the animals was removed, fixed in 10% neutral buffered formalin and paraffin embedded. The livers were stained with H&E and were histologically evaluated. Histological examination of the liver specimens was performed blindly for the presence of hepatic steatosis according to the scoring system designed by the Pathology Committee of the NASH Clinical Research Network, which addresses the full spectrum of lesions of NAFLD (see Kleiner, D. E., Brunt, E. M., Van Natta, M., Behling, C., Contos, M. J., Cummings, O. W., Ferrell, L. D., Liu, Y. C., Torbenson, M. S., Unalp-Arida, A. et al. (2005). Design and validation of a histological scoring system for nonalcoholic fatty liver disease. Hepatology 41, 1313-1321). Images shown were obtained by a Leica ICC50 HD.

Coordination analysis. The Person's correlation R values of each gene with all other genes in the whole transcriptome were calculated in specimens of steatosis vs nonsteatosis, steatosis vs control and nonsteatosis vs control, respectively. The composite correlation (Pc) index was calculated as the Person's R of all R values of each gene in each group combination. This transformation assigned a unique Pc value to each of these genes which reflects the degree by which coordination with the whole transcriptome changes for the corresponding genes of interest. All calculations were conducted with R 3.6.3.

Statistical analysis. For differential expression results were analyzed by pairwise t-test. For correlation studies R value from Pearson's correlation was calculated. In all case results were considered significant when P<0.05.

The following disclosure herein incorporates by reference in its entirety including all supplemental materials the following: Zhang, Chatzistamou, and Kiaris, Transcriptomic coordination at hepatic steatosis indicates robust immune cell engagement prior to inflammation. University of South Carolina. www.kiarislab.com. Correspondence: Hippokratis Kiaris PhD, CLS 713, 715 Sumter Str., Columbia, S.C. 29208-3402 Phone: 803 3611 781 Email: hk@sc.edu. Including but not limited to: Supplementary Table 1. Pc values of NS vs C; Supplementary Table 2. Pc values of S vs C; Supplementary Table 3. Pc values of NS vs S; Supplementary Table 4. GO analyses for transcripts with Pc<−0.2; Supplementary Table 5. GO analyses for transcripts with top 5% cumulative Pc; Supplementary Table 6. GO analyses for differentially expressed genes; and NCBI accession No. of RNAseq data: GSE146846

Figure Legends

FIGS. 6 and 7. Response of deer mice (P. maniculatus) to HFD. a. Body weight in genetically diverse P. maniculatus after administration of regular diet or HFD. Sex, diet and development of steatosis are indicated. Highly variable response was recorded that was not associated with any of the parameters recorded. b. Histopathological appearance of liver sections (H&E) from animals that received regular diet (i) or HFD (ii) but did not develop steatosis or received HFD and developed moderate (iii) or severe (iv) steatosis.

FIG. 8. Pc calculation for the liver transcriptome of P. maniculatus fed with regular diet (C) or HFD and developed (S) or did not develop (NS) steatosis. Scatter plots of Pc versus transcripts are shown in (a), barr plots showing the median values are shown in (b), and box and violin plots depicting Pc distribution are shown in (c). In the left panel results from all genes surveyed are shown while in the right panel only results from common genes in all 3 pairwise comparisons are shown. ****, P<0.00001.

FIG. 9. Number of differentially expressed genes, the volcano plots and the top 3 upregulated and down regulated genes in all 3 pairwise comparisons. FDR cutoff is 0.1 and minimum fold change is 2.

FIG. 10, Table X. Gene Ontology analysis based on Pc data. Genes having Pc<−0.2 were considered. For cumulative Pc analysis, the 3 individual Pc were added and the genes within the 5th percentile of those with higher cumulative Pc were considered.

FIG. 11, Table Y. Gene Ontology analysis based on differential expression. The results for the top 10 processes are shown in the table.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A transcriptome correlation method comprising:
calculating a composite correlation index comprising;
calculating a correlation coefficient value for each transcript with respect to every other transcript in a transcriptome via at least one pairwise comparison:
wherein the composite correlation index indicates either coordination or abolishment of coordination for the at least one pairwise comparison.

2. The method of claim 1, wherein a negative composite correlation index shows an inversed profile of gene coordination.

3. The method of claim 1, wherein a positive composite correlation index shows gene coordination was maintained.

4. The method of claim 1, wherein the composite correlation index shows an extent of transcriptional reprogramming.

5. The method of claim 1, wherein the extent of transcriptional reprogramming indicates a presence of a disease state.

6. The method of claim 5, wherein the disease state is steatosis.

7. The method of claim 1 wherein a composite correlation index indicative value indicates a pro-inflammatory response and transcriptional reprogramming even though no histopathological evidence of inflammation is present.

8. The method of claim 1, further comprising identifying genes exhibiting changes in their transcriptomic profile via calculation of a cumulative composite correlation index.

9. The method of claim 8, wherein the cumulative composite correlation index is calculated via adding independent composite correlation indexes of at least two pairwise comparisons.

10. The method of claim 1 further comprising, calculating a correlation coefficient value for each transcript with respect to every other transcript in a transcriptome via at least three pairwise comparisons:
control versus steatosis;
control versus non-steatosis; and
steatosis versus non-steatosis.

11. An unbiased whole transcriptome analysis comprising:
determining an extent of expression of all transcripts in an organ via coordination analysis;
determining via at least one pairwise comparison an extent of transcriptome reorganization; and
showing engagement of T cell activation to indicate a presence of a disease state.

12. The method of claim 11, wherein the organ is a liver.

13. The method of claim 11, wherein the disease state is steatosis.

14. The method of claim 11, wherein a negative composite correlation index shows an inversed profile of gene coordination.

15. The method of claim 11, wherein a positive composite correlation index shows gene coordination was maintained.

16. The method of claim 11, wherein the composite correlation index shows an extent of transcriptional reprogramming.

17. The method of claim 11, wherein the extent of transcriptional reprogramming indicates a presence of a disease state.

18. The method of claim 11 wherein a composite correlation index indicative value indicates a pro-inflammatory response and transcriptional reprogramming even though no histopathological evidence of inflammation is present.

19. The method of claim 11, further comprising identifying genes exhibiting changes in their transcriptomic profile via calculation of a cumulative composite correlation index.

20. The method of claim 19, wherein the cumulative composite correlation index is calculated via adding independent composite correlation indexes of at least two pairwise comparisons.

21. The method of claim 11 further comprising, calculating a correlation coefficient value for each transcript with respect to every other transcript in a transcriptome via at least three pairwise comparisons:
control versus steatosis;
control versus non-steatosis; and
steatosis versus non-steatosis.
